(19) United States
(12) Patent Application Publication
(10) Pub. No.: US 2026/0215837 A1
(43) Pub. Date: Jul. 30, 2026

(54) MEDICAL DEVICE END CAP WITH ONE OR MORE EXTENDABLE TISSUE MANIPULATION ARMS

(71) Applicant: Boston Scientific Scimed, Inc., Maple Grove, MN (US)

(72) Inventors: Gonzalo Jose SAENZ VILLALOBOS, Alajuela (CR); Scott Edward CORBEIL, Litchfield, NH (US); Jessica BONILLA MORA, Alajuela (CR)

(73) Assignee: Boston Scientific Scimed, Inc., Maple Grove, MN (US)

(21) Appl. No.: 19/454,706

(22) Filed: Jan. 21, 2026

Related U.S. Application Data
(60) Provisional application No. 63/750,477, filed on Jan. 28, 2025.

(51) Int. Cl. A61B 18/14; A61B 1/00; A61B 1/018; A61B 18/00

(52) U.S. Cl. CPC A61B 18/14; A61B 1/00042; A61B 1/00137; A61B 1/018; A61B 2018/00184; A61B 2018/00601; A61B 2018/0091; A61B 2018/00982

(57) ABSTRACT

Medical devices, systems, and related methods for delivering medical tools into a subject (e.g., patient) are described. The medical system includes an insertion device and an end cap device. The end cap device is attachable to a distal portion of the insertion device. The end cap device includes an end cap including a body that defines a lumen aligned with a working channel of the insertion device. The end cap device also includes at least one arm movably coupled to the body. The at least one arm is configured to transition between a retracted position and an extended position, such that when in the extended position, the at least one arm extends radially outward relative to a central longitudinal axis of the lumen.

### CROSS-REFERENCE TO RELATED APPLICATION(S)

This application claims the benefit of priority of U.S. Provisional Patent Application No. 63/750,477, filed on Jan. 28, 2025, the entirety of which is incorporated herein by reference.

### FIELD OF INVENTION

Aspects of this invention relate generally to medical devices for minimally invasive procedures, and more particularly to medical device end caps with one or more extendable arms for tissue manipulation, for example, during submucosal dissection.

### BACKGROUND

Advances in medical devices for performing minimally invasive medical procedures allow a medical professional to visualize internal tissues through small incisions or natural body orifices. For example, endoscopic devices can be used to perform endoscopic submucosal dissection (ESD). However, managing cut or dissected tissue during ESD procedures may present challenges during a medical procedure. For instance, dissected tissue may obstruct views of or impair access to a dissection plane, blood vessels, underlying muscle layer, or otherwise interfere with the procedure, increasing operating time or risk to a patient. Aspects of this disclosure may help manipulate or otherwise manage cut or dissected tissue during a medical procedure.

### SUMMARY

Each of the aspects disclosed herein may include one or more of the features described in connection with any of the other disclosed aspects.

According to one example, a medical system may include an insertion device and an end cap device, and the end cap may be attachable to a distal portion of the insertion device. The insertion device may define a working channel therein. The end cap device may include an end cap that includes a body defining a lumen aligned with the working channel, and at least one arm movably coupled to the body. The at least one arm may be configured to transition between a retracted position and an extended position. In the extended position, the at least one arm may extend radially outward relative to a central longitudinal axis of the lumen.

Any medical device or medical system described herein may include any of the following features. The at least one arm may include a curved shape when in the extended position. The at least one arm may include a plurality of arms arranged circumferentially around the lumen. Each arm of the plurality of arms may be independently movable between the retracted position and the extended position. The body of the end cap may have an opening formed therethrough, and =the at least one arm may be movable through the opening when moving between the retracted position and the extended position. The at least one arm may have a variable cross-section along a longitudinal length of the at least one arm, and at least one distal portion of the at least one arm may be wider than at least one proximal portion of the at least one arm. The at least one arm may be a wire arm formed by a curved section of an actuation wire.

The medical system may include at least one spring coupled to the at least one arm. The at least one spring may be configured to bias the at least one arm toward one of the retracted position or the extended position.

The medical system may include at least one actuation wire coupled to the at least one arm. The at least one actuation wire may be configured to move the at least one arm between the retracted position and the extended position. The medical system may include a pulley positioned distal to a proximal end of the at least one arm, and the at least one actuation wire may interact with the pulley to facilitate movement of the at least one arm. The medical system may include an actuation wire coupled to the at least one arm. The medical system may also include an actuator apparatus coupled to a proximal end of the actuation wire, and the actuator apparatus may be configured to control movement of the at least one arm. The actuator apparatus may include a base having at least one slot. The actuator apparatus may include at least one control lever slidably disposed in the at least one slot. The proximal end of the actuation wire may be movably coupled to the at least one control lever, and a distal end of the actuation wire may be movably coupled to the at least one arm. The at least one control lever may include a plurality of control levers, and the at least one slot may include a plurality of slots configured to guide translation of the plurality of control levers received therein.

The medical system may include a medical device that is insertable through the insertion device and through the end cap device to a target site. The medical device may include a handle having at least one actuator, a shaft extending distally from the handle, and a tool that is movable through the shaft to the target site. The tool may be an electrosurgical knife.

According to another example, a medical system may include an insertion device and an end cap device. The insertion device may define a working channel, and the end cap device may be attachable to a distal end of the insertion device. The end cap device may include an end cap having a body defining a lumen substantially aligned with the working channel. and the end cap device may include a plurality of arms movably coupled to the body. Each arm of the plurality of arms may be independently movable at least between a retracted position and an extended position.

Any medical device or medical system described herein may include any of the following features. The medical system may include an actuator apparatus operably connected to the plurality of arms and configured to independently control movement of each arm. The actuator apparatus may include a plurality of control levers. Each control lever may be associated with one of the plurality of arms, The actuator apparatus may include a plurality of actuation wires, and each actuation wire may be coupled to one of the plurality of control levers, such that movement of the control levers imparts movement to the actuation wires to control a position of the arms.

According to yet another example, a method for performing a medical procedure may include the steps: inserting an insertion device into a body lumen to a target site, inserting a medical device through the insertion device to the target site, cutting a target tissue at the target site via a tool of the medical device, and manipulating the target tissue with an end cap device. Manipulating the target tissue may include distally extending at least one arm of the end cap device from a retracted position toward an extended position distal of an end cap having a body of the end cap device. Distally extending the at least one arm may include independently actuation at least one of a plurality of arms via an actuator apparatus. The actuator apparatus may include a plurality of control levers movably coupled to the plurality of arms.

Any of the examples described herein may have any of these features in combination.

### BRIEF DESCRIPTION OF FIGURES

FIG. 1 illustrates a perspective view of a medical system, according to aspects of the disclosure.

FIG. 2 illustrates a perspective view of an end cap for a medical device, according to an embodiment.

FIGS. 3A and 3B each illustrate a side view of the medical system in various configurations for performing a medical procedure, according to aspects of the disclosure.

FIGS. 4A, 4B, and 4C illustrate perspective views of an end cap having multiple extendable arms, according to aspects of the disclosure.

FIG. 5 illustrates a perspective view of an actuator apparatus, according to aspects of the disclosure.

FIGS. 6A and 6B illustrate another end cap for a medical device, according to aspects of the disclosure.

FIGS. 7A and 7B illustrate yet another end cap for a medical device, according to aspects of the disclosure.

FIGS. 8A, 8B, and 8C illustrate another end cap having a wire arm, according to aspects of the disclosure.

FIGS. 9A, 9B, and 9C illustrate another end cap having a movable wire, according to aspects of the disclosure.

FIGS. 10A, 10B, and 10C illustrate another end cap having a movable arm, according to aspects of the disclosure.

FIG. 11 illustrates a perspective view of another end cap having multiple movable arms, according to aspects of the disclosure.

FIG. 12 illustrates a partial cross-section view of a medical system, according to aspects of the disclosure.

### DETAILED DESCRIPTION

The following description sets forth exemplary aspects of this disclosure. It should be recognized, however, that such description is not intended as a limitation on the scope of this disclosure. Rather, the description also encompasses combinations and modifications to those exemplary aspects described herein.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. Several drawings include arrows labeled “P” and “D,” indicating proximal and distal directions, respectively.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range. As used herein, the phrase “based on” is understood to be equivalent to the phrase “based at least on,” unless indicated otherwise. The term “or” is used disjunctively, such that “at least one of A or B” includes, (A), (B), (A and B), (A or B), (B or A), (A and A), (B and B), etc. It will also be understood that, although the terms first, second, third, etc. are, in some instances, used herein to describe various elements, these elements should not be limited by these terms. These terms are only used to distinguish one element from another. For example, a first arm could be termed a second arm, and, similarly, a second arm could be termed a first arm, without departing from the scope of the various described embodiments. The first arm and the second arm are both arms, but they are not the same arm.

Although scopes and endoscopes are referenced herein for illustration purposes, it will be appreciated that the disclosure encompasses any suitable medical device configured to allow an operator to access and view internal body anatomy of a subject and/or to deliver medical instruments. As such, aspects of this disclosure include insertion or medical devices that may be used to deliver one or more of, for example, biopsy forceps, graspers, baskets, snares, probes, scissors, capture/retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device may be used in connection with the features described herein.

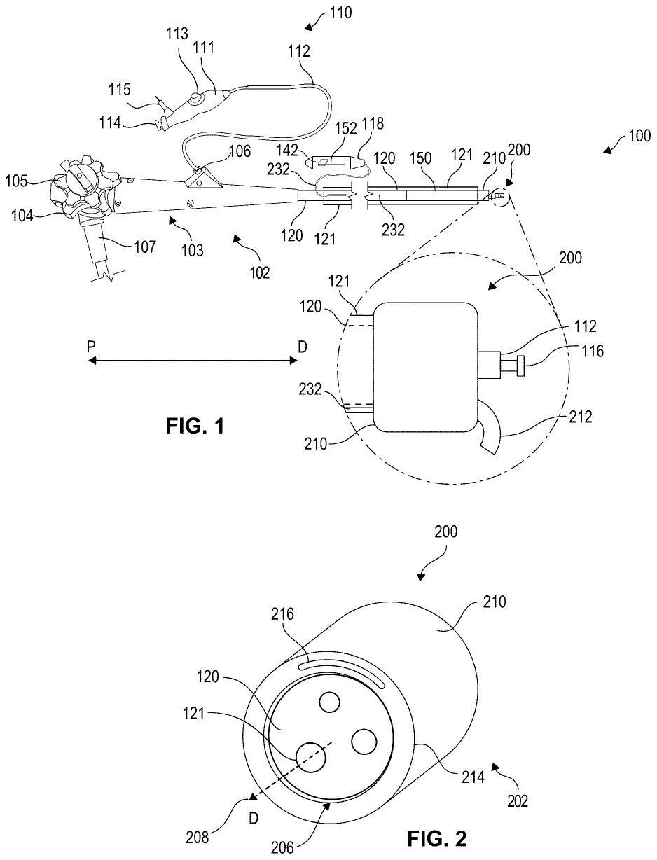

FIG. 1 illustrates a perspective view of a medical system 100. Medical system 100 may include an insertion device 102 and a medical device 110. Insertion device 102 includes a shaft 120 and a handle 103. Handle 103 may be attached to the proximal end of shaft 120. Handle 103 may feature several adjustment knobs or controls 104 and 105, which may allow for manipulation of insertion device 102. Handle 103 may have one or more ports 106 in communication with a working channel, for example, extending through a portion of 103 and shaft 120. Handle 103 may include an umbilicus 107, which may allow power transmission and/or data transmission for medical system 100. Alternatively or additionally, umbilicus 107 may provide one or more fluids or suction to insertion device 102.

Medical device 110 may include a handle 111 and a shaft 112, with shaft 112 extending distally from handle 111. A trigger 113 may be positioned on handle 111, and trigger 113 may be used to actuate (e.g., energize, extend, retract, etc.) one or more portions of medical device 110. Medical device 110 may also feature a fluid port 114 and an electrical hub 115, which may provide respectively allow for fluid delivery and electrical connections to medical device 110. As discussed in detail below, a distal end of shaft 120 includes or is otherwise coupled to an end cap device 200, which includes an end cap body 210 and one or more extendable or otherwise movable arms 212, In some examples, a user (e.g., medical professional) may be able to manipulate cut or severed portions of tissue at a target site during a medical procedure via one or more portions of end cap device 200.

Insertion device 102 may be an endoscope. However, as mentioned above, insertion device 102 may be any other insertion or medical device. Insertion device 102 includes shaft 120 extending from a proximal end to a distal end. Shaft 120 of insertion device 102 may have a segmented or articulated design, which may allow for flexibility and maneuverability during use, for example at or proximate the distal end of shaft 120. For example, insertion device 102 may include an articulation section 150. As mentioned, end cap device 200 may be attached to a distal portion of insertion shaft 120, for example, at the distal end having articulation section 150. End cap device 200 includes end cap body 210, which may be coupled to the distal portion of insertion shaft 120. End cap device 200 also includes one or more extendable arms 212, which may be movable (e.g., extendable and retractable) relative to end cap body 210, for example, to help manipulate tissue.

In some aspects, medical system 100 may include one or more control handles 118 coupled to end cap device 200, for example, movably coupled via one or more actuation wires 232. In some examples, actuation wire 232 may extend distally from control handle 118 toward the distal end of insertion shaft 120, such that actuation wire 232 is external to the channels or lumens defined through shaft 120. For example, medical system 100 may include a sheath 121 defining a lumen therein, which may be shaped and sized to enclose at least respective portions of insertion shaft 120 and actuation wire 232 therein (e.g., radially enclose in part or whole). In other examples, although not shown, medical system 100 may include a plurality of sheaths or tubes defining a plurality of lumens, which may be shaped and sized to respectively enclose one or more actuation wires therein. In alternative implementations, these control wires may distally extend from control handle 118 through one or more channels (e.g., the working channel or another lumen) defined in insertion shaft 120 toward the distal end having end cap device 200, such that distal portions of actuation wire 232 are movably attachable to extendable arms 212.

In some aspects, control handle 118 may include one or more control levers 142 coupled to one or more actuation wires, such that actuation or movement of a respective control lever 142 causes actuation or movement of a respective actuation wire 232. In turn, actuation via control handle 118 may allow the user to move one or more extendable arms 212 between retracted and extended positions. For example, control handle 118 may have one control lever 142 slidably disposed in a slot 152 formed therein and movably coupled to a proximal end of actuation wire 232, such that sliding the one control lever 142 causes one extendable arm 212 to move, for example, from a retracted position to an extended position (e.g., distally away from body 210). In other examples, although not shown, medical system 100 may include one or more foot pedals having one or more control levers coupled to one or more actuation wires, such that actuation or movement of a respective foot pedal causes actuation or movement of a respective actuation wire.

In other aspects, control handle 118 may be integrated into a handle or other proximal components of medical system 100. In some examples, control handle 118 may be integrated into insertion device 102 and/or medical device 110, such as integrated into one of handle 103 and/or handle 111. In other examples, control handle 118 may be attachable to insertion device 102 and/or medical device 110 via one or more fasteners.

FIG. 2 illustrates a perspective view of end cap device 200, according to some aspects of this disclosure. End cap device 200 includes an end cap 202 having end cap body 210 defining a lumen 206 therein, for example extending along a central longitudinal axis 208 of end cap device 200. Lumen 206, for example, a proximal portion of lumen 206, may be configured to help couple end cap body 210 to the distal end of insertion shaft 120. Additionally, lumen 206, for example, a distal portion of lumen 206, may be configured to receive one or more medical devices, tools, or instruments. For example, lumen 206 may receive one or more portions of medical device 110, for example, when shaft 112 of medical device 110 is delivered through a working channel of insertion shaft 120. In some aspects, lumen 206 may be shaped and sized to help guide medical tools along a designated path, for example, to help reduce risk of internal collision between two or more medical tools therein. In some examples, one or more inner surfaces defining lumen 206 may be coated with a low-friction material, for example to reduce resistance when advancing or retracting medical tools.

End cap device 200 may include one or more extendable arms disposed at least partially within body 210, for example, extendable arm 212 (FIG. 1). Extendable arm 212 may be movable between a retracted position (e.g., contained within body 210) and an extended position (e.g., at least partially distally extended from body 210 relative to central longitudinal axis 208). In some cases, extendable arm 212 may be made of shape memory materials, such as Nitinol or other nickel-titanium alloys, which may allow extendable arm 212 to be pre-formed with a specific shape (e.g., curved shaped) such that extendable arm 212 may return to said specific shape when extended, regardless of its compressed state within body 210.

End cap 202 may include one or more openings 216 formed through one or more surfaces. For example, a distal opening 216 may be formed in a distal surface 214 of body 210, with the distal opening being configured to receive extendable arm 212 therethrough. As shown in FIG. 1, extendable arm 212 is in a partially extended position, protruding distally from body 210 relative to central longitudinal axis 208. In some cases, distal opening 216 may be oriented approximately parallel to central longitudinal axis 208, such that extendable arm 212 may be positioned approximately parallel with central longitudinal axis 208. In these aspects, end cap device 200 may be configured to allow for tissue manipulation during a medical procedure, for example, to help remove tissue from the target site, help allow passage or delivery of one or more other instruments (e.g., an electrosurgical knife, a grasper, a basket, a cautery device, etc.), help prevent tissue (e.g., cut tissue) from impairing the visualization of one or more cameras or the illumination of the target site by one or more illumination device, or otherwise help in the medical procedure. In some examples, extendable arm 212 may have one or more tissue-contacting surfaces, which may incorporate micro-texturing or small protrusions to facilitate manipulation of tissue during the medical procedure. These features may vary in size, shape, or pattern to optimize traction for different types of tissue. For example, one or more surfaces of extendable arm 212 may have micro-texturing that includes a series of ridges, grooves, dimples, or a combination thereof arranged in regular or irregular patterns.

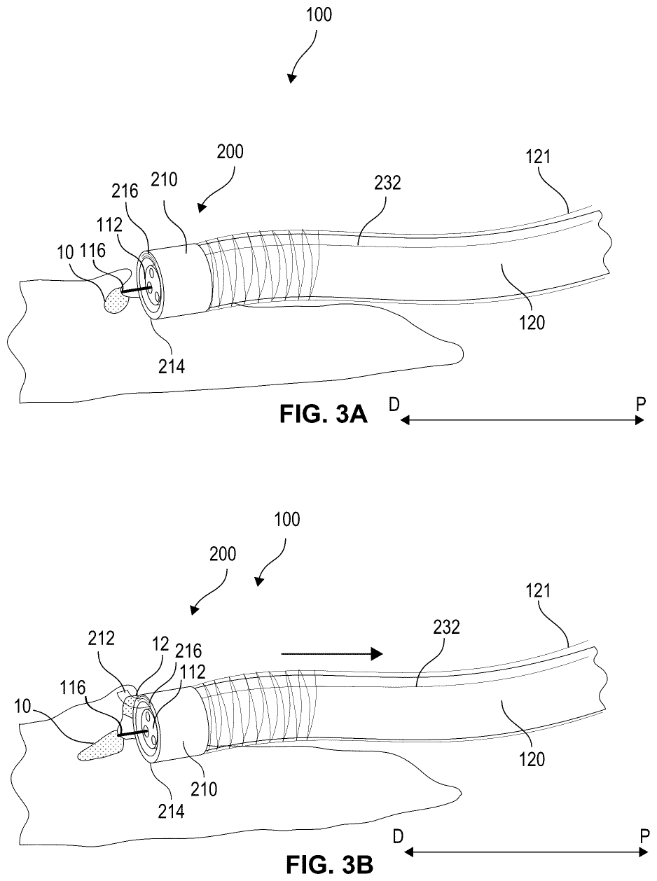

FIGS. 3A and 3B illustrate end cap device 200 in use for tissue manipulation during a medical procedure. FIG. 3A depicts medical system 100 with arm 212 of end cap device 200 in a retracted position (e.g., for insertion to a target site having tissue 10), and FIG. 3B shows arm 212 of end cap device 200 in the extended position (e.g., for manipulation of cut or severed portion 12 of tissue 10). Inserting end cap device 200 while arm 212 is in the retracted position may allow for precise positioning at the target site (e.g., to cut tissue 10 via tool 116). After cutting tissue, the user may manipulate end cap device 200, such that arm 212 transitions from the retracted position to an at least partially extended position. Manipulating end cap device 200 may include, for example as shown, manipulating (e.g., proximally pulling) one or more actuation wires 232 movably coupled to extendable arm 212. In this aspect, arm 212 may contact, push, move, or otherwise manipulate one or more cut portions 12 of tissue 10 as extendable arm 212 moves distally toward the extended position (e.g., through distal opening 216 formed in distal surface 214 of body 210). While end cap device 200 is shown as having single extendable arm 212, in other implementations (e.g., as discussed below), end cap device 200 may include multiple extendable arms 212. In other aspects, medical system 100 may include a sheath 121 or the like defining a lumen therein, for example, which receives/contains shaft 120 and actuation wire 232 therein.

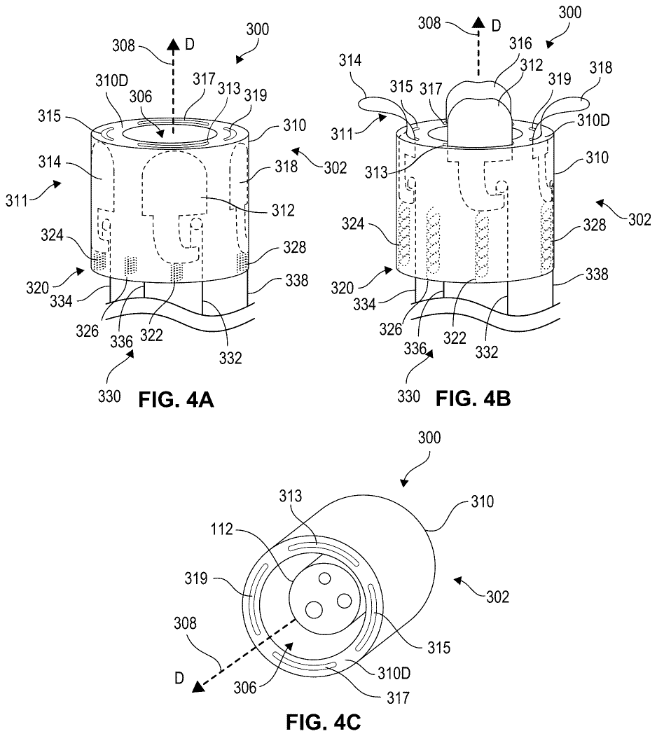

FIGS. 4A, 4B, and 4C illustrate an end cap device 300, including a plurality of extendable arms 311, according to aspects of the disclosure. As shown, end cap device 300 includes an end cap 302 having a body 310 defining a lumen 306 extending along a central longitudinal axis 308 of end cap device 300. End cap device 300 may be coupled to a distal portion of a medical device (not shown), for example coupled to the distal portion of shaft 120 (FIG. 1), for example to manipulate cut portions 12 of tissue 10 using one or more of arms 311, as discussed above with respect to FIGS. 3A and 3B. As discussed herein, end cap 302 may define a plurality of slots that are configured to receive one of arms 311 therein. For example, the plurality of slots may be defined through a distal surface 310D of body 310 approximately parallel to lumen 306. End cap device 300 may also include a plurality of springs 320 configured to bias arms 311 relative to body 310, and a plurality of wires 330 movably coupled to arms 311. As further discussed, a user may independently or simultaneously actuate (e.g., push or push) wires 330 during a medical procedure, which in turn may distally urge arms 311 from respective slots in body 310 toward the extended position, and after releasing wires 330, compressive force from springs 320 may proximally urge arms 311 toward the retracted position.

In some aspects, arms 311 comprises a first arm 312 positioned in a first slot 313 defined in body 310, a second arm 314 positioned in a second slot 315 defined in body 310, a third arm 316 positioned in a third slot 317 defined in body 310, and a fourth arm 318 positioned in a fourth slot 319 defined in body 310. Arms 311 may be arranged may be arranged to radially surround lumen 306 (e.g., approximately parallel to central longitudinal axis 308), for example such that radial distribution of arms 311 may allow for comprehensive coverage of the target site and thus allow simultaneous tissue manipulation in multiple directions. This multi-arm configuration may enhance versatility during medical procedures, enabling more efficient and precise tissue handling while maintaining a clear passage for medical tools, such as tool 116 (FIG. 1).

In some aspects, arms 311 may be independently or simultaneously movable between retracted and extended positions, such that arms 311 independently movable through a respective slot relative to body 310. For example, first arm 312 may independently move between the retracted position contained within first slot 313 and the extended position distal of body 310 relative to central longitudinal axis 308. Similarly, second arm 314 may be independently movable through second slot 315, third arm 316 may be independently movable through third slot 317, and fourth arm 318 may be independently movable through fourth slot 319.

In FIG. 4A, arms 311 are shown retracted, such that each of arms 311 may be substantially flush with or recessed within a distal surface 310D of body 310. This retracted position may provide a streamlined profile for end cap device 300 (e.g., for insertion to a target site during the medical procedure). FIG. 4B depicts arms 311 in an extended position, at least partially protruding outward from distal surface 310D of body 310. When in the extended position, each of arms 311 may have a curved shape, for example extending radially away from central longitudinal axis 308. FIG. 4C provides an end perspective view of end cap device 300, illustrating one exemplary arrangement of arms 311 disposed within body 310 around lumen 306. As shown, the plurality of slots may be arranged circumferentially around distal surface 310D relative to central longitudinal axis 308, such that arms 311 received therein are similarly arranged.

As shown in FIGS. 4A and 4B, end cap device 300 may include one or more biasing members, for example, springs 320 disposed in respective slots for biasing respective one of arms 311 toward the retracted position. In the example shown, springs 320 includes a first spring 322 coupled to first arm 312, a second spring 324 coupled to second arm 314, a third spring 326 coupled to third arm 316, and a fourth spring 328 coupled to fourth arm 318. Although third arm spring 326 is shown, the third arm 316, third pulley, and distal portion of third wire 336 are omitted for clarity. Springs 320 may include two springs, or three or more springs (not shown). Springs 320 may be respectively positioned within walls of body 310 and may provide a biasing force for arms 311, for example, toward the retracted position. Each of springs 320 may be secured to one or more inner surfaces defined by body 310, for example respectively secured within one of the slots therein. For example, first spring 322 may be secured to inner surface of first slot 313, second spring 324 may be secured to inner surface of second slot 315, third spring 326 may be secured to inner surface of third slot 317, and fourth spring 328 may be secured to inner surface of fourth slot 319. In some cases, springs 320 may be compression springs, tension springs, torsion springs, or a combination thereof. For example, first spring 322 may be a tension spring having a proximal end secured to inner surface of first slot 313 and a distal end secured to first arm 312, such that moving first arm 312 towards the extended position causes first spring 322 to extend distally within first slot 313 and store force to return first 312 to retracted position (e.g., once wires 330 is released). The spring constant of each of springs 320 may be tailored to provide optimal force for retraction while allowing for extension when actuated via wires 330.

As further shown, wires 330 may be movably coupled with arms 311, for example, plurality of wires 330 may include a first wire 332 movably coupled to first arm 312, a second wire 334 movably coupled to second arm 314, a third wire 336 movably coupled to third arm 316, and a fourth wire 338 movably coupled to fourth arm 318. Plurality of wires 330 may be connected to arms 311, for example, to facilitate extension of respective arms when the respective wire is actuated.

As discussed, each of arms 311 may be independently controllable, which may help to allow for complex or varied tissue manipulations. For example, first arm 312 may be extended to manipulate tissue while the other three arms of arms 311 respectively retain the retracted position. In another example, each of arms 311 may be extended to different extents relative to other arms 311. For instance, first arm 312 may be fully extended, second arm 314 may be fully retracted, third arm 316 may be partially extended, and fourth arm 318 may be fully retracted. It should be understood that other arrangements, or combinations of arm arrangements, are contemplated within the scope of this disclosure. Control of individual arms 311 may be achieved through various mechanisms. In some aspects, a separate actuation device may be used to manipulate each arm independently. Alternatively, actuators may be integrated into a handle or other component of the medical device to which end cap device 300 is attached. These actuators may be designed to allow precise control over the extension and retraction of each arm, potentially enabling the operator to perform intricate tissue manipulations during medical procedures.

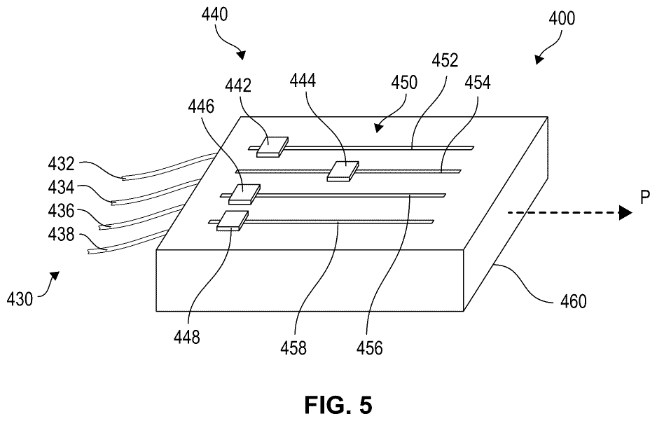

Referring now to FIG. 5, an actuator apparatus 400 for individually manipulating extendable arms of an end cap device according to some aspects is shown. In some examples, actuator apparatus 400 may be configured to control arm 212 of end cap device 200 similar to control handle 118 (FIGS. 1-2), or actuator apparatus 400 may be configured to control multiple arms 311 of end cap device 300 (FIGS. 4A-4C), via independent control and/or simultaneous control. Actuator apparatus 400 may include a base 460 having a plurality of slots 450 formed through a surface of base 460. Actuator apparatus 400 may include a plurality of control elements or control levers 440 respectively positioned within one of slots 450 and respectively coupled to one of a plurality of actuation wires 430. A user (e.g., medical professional) may move or slide control levers 440 within slots 450, which in turn causes at least one of actuation wires 430 to move or slide in a same direction. Movement of at least one actuation wire, in turn, actuates or otherwise controls at least one extendable arm of at least one end cap movably coupled to distal ends of actuation wires 430.

In the example shown, actuation wires 430 includes a first wire 432, a second wire 434, a third wire 436, and a fourth wire 438. Control levers 440 includes a first lever 442, second lever 444, a third lever 446, and a fourth lever 448. Slots 450 includes a first slot 452, a second slot 454, a third slot 456, and a fourth slot 458. Each of actuation wires 430 has a proximal portion of wire extending through a side surface of base 460 and extending at least partially through one of slots 450 for coupling with one of control levers 440. For instance, a proximal portion of first wire 432 extends into base 460, through first slot 452, and is movably coupled with first lever 442. As the user slides first lever 442 along first slot 452 (e.g., translation, proximally/distally), first wire 432 moves through and relative to base 460 which in turn causes actuation of a first extendable arm, such as first arm 312 of end cap device 300 (FIGS. 4A-4C). Similarly, a proximal portion of second wire 434 may extend through second slot 454 and be coupled to second lever 444. A proximal portion of third wire 436 may extend through third slot 456 and be coupled to third lever 446. A proximal portion of fourth wire 438 may extend through fourth slot 458 and be coupled to fourth lever 448. In one example, actuation wires 430 may correspond to actuation wires 330. In these aspects, actuator apparatus 400 may be used to control end cap device 300 to perform the medical procedure by independently actuating of one or more of arms 311 of end cap device 300 (FIGS. 4A-4C) via sliding one or more of control levers 440 of actuator apparatus 400.

In some aspects, distal ends of actuation wires 430 may be directly or indirectly coupled to a plurality of extendable arms, such as arms 311 (FIGS. 4A-4C). In some cases, actuation wires 430 are directly connected with respective arms via mechanical or chemical bonding (e.g., soldering, welding, crimping, etc.). In other cases, a coupler or other mechanism may be used for coupling actuation wires 430 with one or more extendable arms, for example, linkages or pulleys may be used to impart movement from actuation wires 430 to one or more arms. As such, actuator apparatus 400 may be configured to allow independent control of each arm of an end cap having multiple arms, and therefore allow for relatively complex tissue manipulations or patterns during medical procedures.

In some aspects, slots 450 may be linear slots or openings formed along an extent of upper surface of base 460, for example as shown in FIG. 5. In some cases, slots 450 may be have different shapes or patterns formed in base 460, for example such that control levers 440 are movable along base 460 in one or more non-linear patterns (e.g., corresponding to non-linear actuation of arms). In another implementation, slots 450 may be positioned on two or more surfaces of base 460, for example on opposing surfaces of base 460. In some implementations, slots 450 may have one or more lateral extensions or indentations, for example, which may allow the user to releasably lock control levers 440 relative to respective slots 450, such that actuation wires 430 are secured relative to the base 460 and respective extendable arms are locked in desired position (e.g., extended position). In some cases, actuator apparatus 400 may also include one or more biasing elements, such as springs, to bias control levers 440 proximally or distally relative to respective slots 450, for example, so actuator apparatus 400 may bias at least one extendable arm toward the retracted position.

In other aspects, actuator apparatus 400 may be integrated into a handle or other proximal components of a medical device or system. In some examples, actuator apparatus 400 may be integrated into insertion device 102 and/or medical device 110, such as integrated into one of handle 103, handle 111, and/or control handle 118 (FIG. 1). In other examples, actuator apparatus 400 is independent of insertion device 102 and medical device 110. In yet another example, actuator apparatus 400 may be attachable to insertion device 102 and/or medical device 110 via one or more fasteners.

FIGS. 7A-7B illustrate another end cap device 500 for a medical device, according to aspects of the disclosure. As shown, end cap device 500 may include an end cap 502 having a body 510 defining a lumen 506 extending therethrough along a central longitudinal axis 508 of end cap device 500. End cap 502 may define a slot 513 extending therethrough, for example, extending through a distal surface 510D of body 510 parallel to central longitudinal axis 508. End cap device 500 may include an extendable arm 512 configured to transition between a retracted position (FIG. 7A) and an extended position (FIG. 7B), for example through slot 513 in response to movement of an actuation wire 532 having a distal portion of wire movably coupled to extendable arm 512.

As seen in FIGS. 7A-7B, end cap device 500 may include a pulley 533, for example movably coupled to a portion of extendable arm 512. Pulley 533 may be configured to allow extendable arm 512 to move between the retracted position and extended position. For example, in response to one of pushing (distally) or pulling (proximally) of actuation wire 532, extendable arm 512 may be configured to move from one of the retracted or extended positions to another one of the retracted or extended positions.

Actuation wire 532 may traverse pulley 533 to move extendable arm 512 between the retracted and extended positions. In some aspects, extendable arm 512 may have a curved portion or widened portion, for example a proximal portion 512P of arm 512 that has more curvature and/or wider than a distal portion 512D of arm 512. Proximal portion 512P of arm 512 (e.g., curved portion or widened portion) may be coupled to distal portion of actuation wire 532. The shape and size of proximal portion 512P may allow for coupling of arm 512, pulley 533, and actuation wire 532. For example, movement of wire 532 may cause movement of arm 512 via pulley 533, such that distal portion 512D of arm 512 is distally urged through slot 513 of body 510 toward the extended position. In some aspects, when arm 512 is fully extended as shown in FIG. 7B, proximal portion 512P of arm 512 may abut pulley 533, for example, to inhibit further distal movement and retain arm 512 therein (e.g., forming a stop surface). In some examples, slot 513 may be shaped and sized to contact distal portion 512D of arm 512, such that further distal movement of arm 512 is inhibited by geometry of slot 513 when arm 512 is fully extended. For instance, arm 512 may have a tapered outer surface that allows distal portion 512D to be distally urged through slot 513 and into contact with slot 513 at the fully extended position. In other implementations, body 210 may define one or more protrusions (not shown), for example, located proximate distal surface 510D and extending radially into slot 513.

End cap 502 may also include one or more springs positioned within body 510. For example, as shown in FIGS. 7A-7B, end cap 502 may include a spring 522 coupled to extendable arm 512 (e.g., to proximal portion 512P) and secured to one or more portions of body 510, for example, such that spring 522 is configured to bias extendable arm 512 towards the retracted position or extended position. In this example, spring 522 is configured to bias extendable arm 512 toward the retracted position, such that proximally pulling actuation wire 532 causes arm 512 to move distally relative to body 510 and that releasing actuation wire 532 after proximal movement causes spring 522 to bias arm 512 toward the retracted position. In other implementations, spring 522 may be configured to help increase resistance or resistive force to distal movement of arm 512 relative to body 510. As such, movement of extendable arm 512 may be controlled by the interaction between actuation wire 532, pulley 533, and spring 522, which can adjusted or customized to accommodate specific needs of the user or medical procedure.

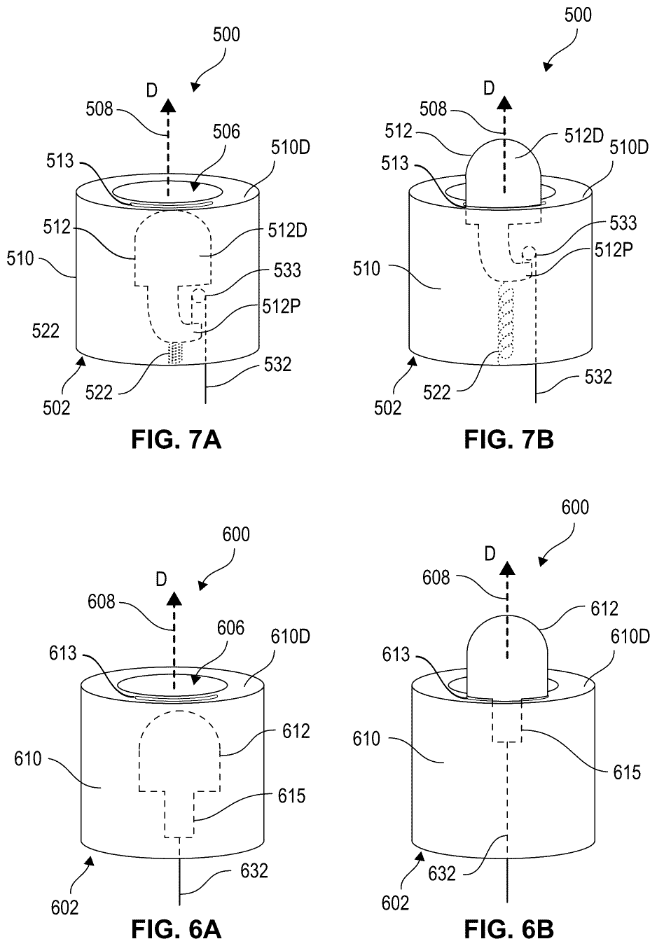

FIGS. 6A-6B illustrate another end cap device 600, including an end cap 602 having a body 610 defining a lumen 606 extending therethrough along a central longitudinal axis 608. End cap device 600 may include one or more extendable arms 612 that are movably coupled to end cap 602. In the example shown, arm 612 is movably disposed within a slot 613 formed in a distal surface 610D of body 610, allowing for transition between a retracted position (FIG. 6A) and an extended position (FIG. 6B).

In some aspects, a size and contour of arm 612 may enhance tissue manipulation capability of end cap device 600. In the example shown, end cap device 600 includes arm 612 having a proximal portion 615 and a distal portion, with proximal portion 615 being narrower than the distal portion. In some cases, having the wider distal portion of arm 612 may allow for greater surface area contact with tissue, for example, which may help to improve grip or retraction capability of end cap device 600 during the medical procedure. This may also be useful, for example, to define a contact surface for arm 612 to engage or abut body 610, such as to inhibit proximal translation beyond a designated position.

Arm 612 may be coupled to one or more actuation wires 632, with wire 632 extending through a proximal portion of body 610 and movably coupled to proximal portion 615 of arm 612. For example, wire 632 may include a proximal portion, which may be coupled to a handle or other device (e.g., actuation apparatus 400), and may also include a distal portion that extends through a proximally facing surface of body 610 for attachment to arm 612. In these aspects, moving the proximal portion of wire 632 causes arm 612 to move between the retracted and extended positions. For example, distal movement of wire 632 may impart distal movement to arm 612 (e.g., toward the extended position shown in FIG. 6B), and proximal movement of wire 632 may impart proximal movement to arm 612 (e.g., toward the retracted position shown in FIG. 6A). In these examples, arm 612 may translate proximally and distally between retracted and extended positions, respectively.

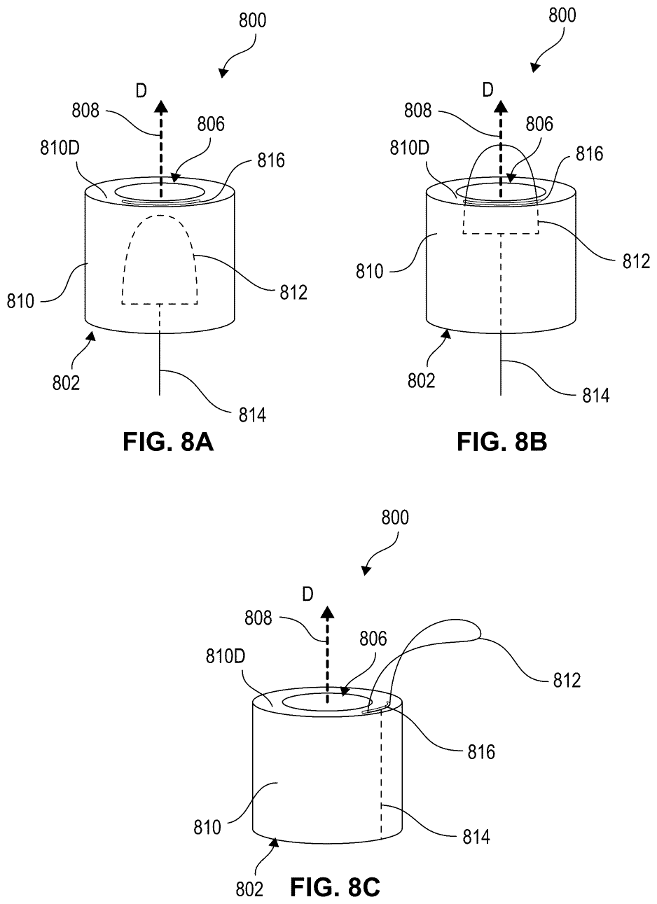

FIGS. 8A-8C illustrate end cap device 800, including an end cap 802 and an actuation wire 814 having a wire arm 812. End cap 802 may have a cylindrical body 810 defining a lumen 806 extending therethrough along a central longitudinal axis 808 of end cap device 800. Actuation wire 814 may have a curved or shaped distal portion of wire defining wire arm 812. Body 810 may define one or more slots configured to receive wire arm 812, for example, a slot 816 formed in a distal surface 810D of body 810 and extending approximately parallel to central longitudinal axis 808.

In some aspects, wire arm 812 may be movably disposed within slot 816 and configured to transition between various positions or configurations. FIGS. 8A-8C illustrate one example of end cap device 800 as wire arm 812 moves between various positions relative to body 810. FIG. 8A depicts one example of wire arm 812 in a fully retracted position. FIG. 8B illustrates one example of wire arm 812 in a partially extended position, and FIG. 8C shows a perspective view of one example of wire arm 812 in a fully extended position through slot 816 distal of body 810. The extent that wire arm 812 extends from end cap body 810 while in the fully extend position may be determined on a length of actuation wire 814.

The transition of wire arm 812 between these positions may be facilitated by movement of actuation wire 814. For example, distally pushing actuation wire 814 may cause wire arm 812 to extend in a distal direction through slot 816, whereas proximally pulling actuation wire 814 may retract wire arm 812 in a proximal direction toward slot 816. In some examples, slot 816 may be positioned on a distalmost surface of body 810, which may allow for deployment angle of wire arm 812 in the distal direction, or substantially distal direction, relative to body 810. In some examples, slot 816 may have curved profile or surfaces to guide wire arm 812 along specific deployment path. In some implementations, body 810 may have two or more slots formed therein, for example at two or more angles on distal surface of body 810, providing two or more options for deploying wire arm 812 at various predetermined angles. Additionally, in some implementations, the distal surface of body 810 may have various shapes, such as convex or concave curvature, which may urge wire arm 812 along a particular path or direction. In other implementations, for example, to help enhance stability and control, end cap device 800 could having guide rail component positioned adjacent slot 816 to help the movement of guide wire arm 812 between the retracted position and the extend position.

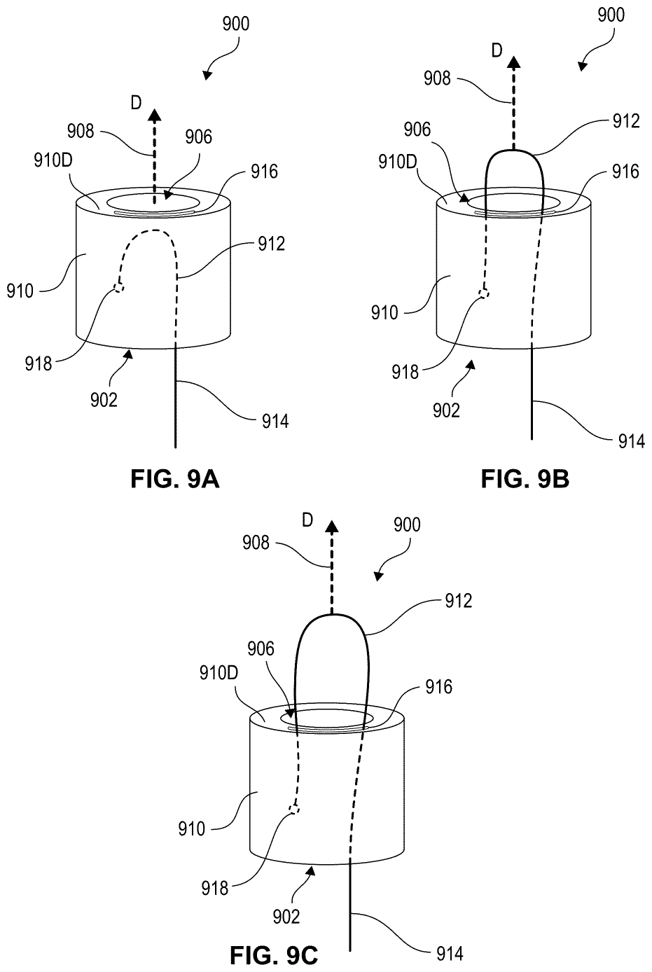

FIGS. 9A-9C illustrate an end cap device 900, including an end cap 902 that has a body 910 defining a lumen 906 therethrough. End cap device 900 includes at least one wire arm 912 that is movable through and relative to body 910 relative to a central longitudinal axis 908 defined through lumen 906. As shown, the at least one wire arm 912 may be secured within body 910 at an attachment point 918, which may serve as a fixed end or fixed portion (e.g., distal end or distal portion) of an actuation wire 914. As shown, the distal portion of actuation wire 914 defines wire arm 912, such that attachment point 918 may allow wire arm 912 to move through and relative to body 910 in response to actuating a proximal portion of actuation wire 914, for example, pushing in a distal direction. Attachment point 918 may be positioned parallel to central longitudinal axis 908, such that wire arm 912 extends and retracts in a direction approximately parallel to central longitudinal axis 908. For example, body 910 may include a distally-facing surface 910D having a slot 916 formed therein, such that wire arm 912 slides through and relative to slot 916.

FIG. 9A shows wire arm 912 in a retracted position (e.g., enclosed within end cap 902). FIG. 9B shows wire arm 912 in a partially extended position (e.g., partially enclosed within end cap 902) and protruding distally from surface 910D through slot 916, and FIG. 9C shows wire arm 912 in a fully extended position (e.g., not contained within end cap 902). As discussed, attachment point 918 may fix the distalmost portion of actuation wire 914 to end cap 902, such that wire arm 912 can be pushed or pulled through slot 916. Although slot 916 is shown on the distalmost surface of end cap 902, it should be understood that end cap 902 may have different configurations with different slots formed therein. In other implementations, end cap device 900 may include two or more fixation locations.

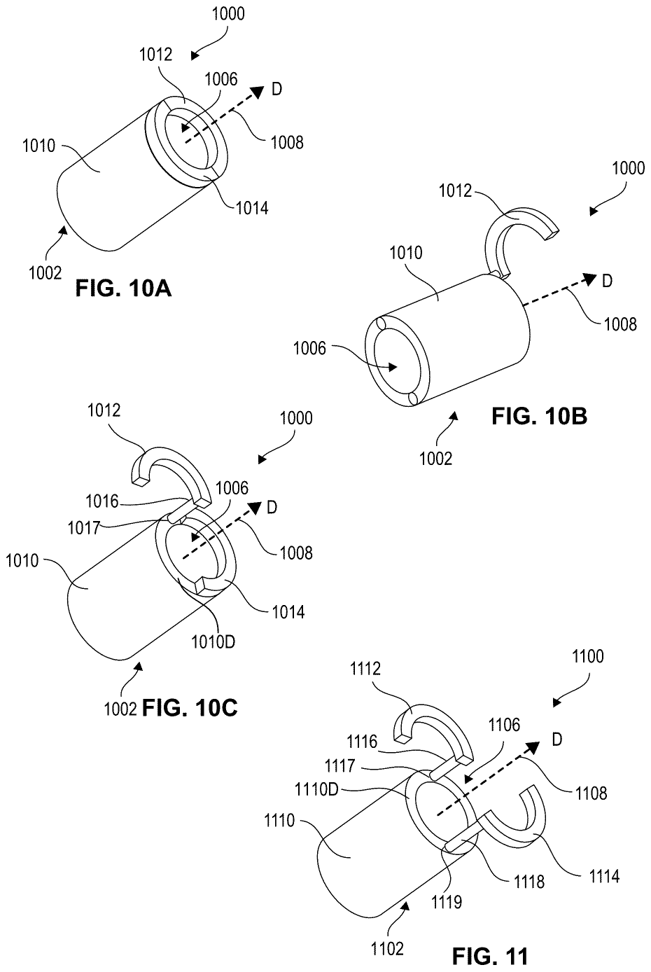

FIGS. 10A-10C illustrate various views of an end cap device 1000, according to other aspects of this disclosure. End cap device 1000 may include an end cap 1002 having a body 1010 defining a lumen 1006 therein, for example, along a central longitudinal axis 1008 defined through a center of lumen 1006 defined through a distalmost surface 1010D of body 1010. End cap device 1000 may be attached to a distal end of an insertion device to support tissue manipulation during a medical procedure via one or more extendable arms. In the example shown, end cap device 1000 includes a first arm 1012 and a second arm 1014, at least one of which is movable relative to body 1010 of end cap 1002. In some examples, at least one of first arm 1012 and second arm 1014 may be movable relative to body 1010 between a retracted position (e.g., positioned flush with distalmost surface 1010D of body 1010) and an extended position (e.g., positioned distally and/or radially outward from body 1010). For example, first arm 1012 may be extendable between the retracted position shown in FIG. 10A, and the extended position shown in FIGS. 10B and 10C, while second arm 1014 may be stationary relative to body 1010.

As the perspective view of FIG. 10C illustrates, end cap device 1000 may include a shaft 1016 pivotably coupled to first arm 1012. End cap 1002 may define one or more channels 1017 shaped and sized to receive shaft 1016 therethrough, for example to pivotably couple first arm 1012 and end cap 1002. Shaft 1016 may translate distally and/or proximally through and relative to end cap 1002. For example, shaft 1016 may be slidably disposed in channel 1017 and movably coupled to one or more wires (not shown). Shaft 1016 may also be rotatable about a longitudinal axis thereof, for example in response to rotation of the actuation wire(s) coupled to shaft 1016. Translating and/or rotating shaft 1016 may, in turn, cause translation and/or rotation of first arm 1012 relative to end cap 1002. In some implementations, although not shown, second arm 1014 may also be movable relative to end cap body 1010 via a respective shaft or wire.

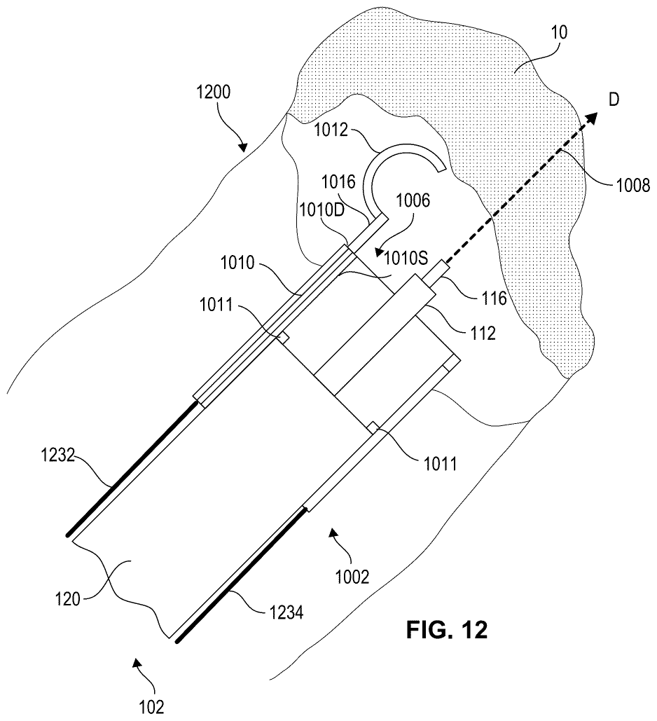

FIG. 12 illustrates an exemplary medical system 1200, including end cap 1002 in FIGS. 10A-10C and attached to distal portion of insertion device 102 in FIG. 1. Medical system 1200 also includes shaft 112 extending through end cap 1002 and tool 116 extending through shaft 112. As mentioned above, tool 116 may be an electrosurgical knife for cutting tissue 10 during a medical procedure. Actuating end cap device 1000 can be useful to deflect or otherwise manipulate tissue 10, such as manipulation of tissue 10 that has not been cut and/or cut portions 12 of tissue 10 (FIG. 1). For example, actuation of a first wire 1232 movably coupled shaft 1016 may cause first arm 1012 to move relative to end cap body 1010, e.g., proximally and distally between the retracted and extended positions, respectively.

In some aspects, medical system 1200 may include a sheath, tube, or the like (not shown) defining a lumen therein that is shaped and sized to receive shaft 120, first wire 1232, and second wire 1234 therein. The sheath may radially surround, partially or wholly, for example to safely deliver first wire 1232 and/or second wire 1234 to the target site (e.g., without causing damage to subject anatomy). As such, the sheath may extend along an external surface of insertion device 102, for example distally from handle 103 along the external surface of shaft 120 and/or articulation section 150 (FIG. 1).

In other aspects, end cap device 1000 may be attached to the distal portion of insertion device 102 via one or more connectors, for example via friction fit, snap-fit, threaded connection, magnetic coupling, and/or stop surfaces. In the example shown in FIG. 12, end cap device 1000 includes at least one stop surface 1011 secured to or formed by an inner surface 1010S of body 1010 and configured to contact a distalmost surface or portion of shaft 120. In some examples, stop surface 1011 includes a ring or other protrusion extending radially inward from inner surface 1010S of body 1010 toward central longitudinal axis 1008. Stop surface 1011 may be positioned at different locations of end cap device 1000, for example, proximate of a distally facing surface 1010D of body 1010. In some examples, end cap device 1000 may include a low-friction bearing surface (e.g., inner surface 1010S) or lubricous coating to facilitate movement (e.g., rotation, translation) of end cap 1002 relative to the distal portion of insertion device 102 (e.g., shaft 120).

FIG. 11 illustrates a perspective view of another end cap device 1100, according to yet another aspect of this disclosure. As shown, end cap device 1100 includes an end cap 1102 having a body 1110 defining a lumen 1106 extending therein along a central longitudinal axis 1108 of end cap device 1100. End cap device 1100 also includes a first shaft 1116 movably coupled to a first arm 1112, and a second shaft 1118 movably coupled to a second arm 1114. First shaft 1116 may be slidably disposed in a first channel 1117 defined by body 1110, and second shaft 1118 may be slidably disposed in a second channel 1119 defined by body 1110. For example, first channel 1117 and second channel 1119 may respectively extend approximately parallel to central longitudinal axis 1108. Shown in their extended position, first shaft 1116 and second shaft 1118 may define pivot points for first arm 1112 and second arm 1114, respectively, such that a user may for example use a first wire to control first shaft 1116 and a second wire to control second shaft 1118. Movement (e.g., translation and/or rotation) of first shaft 1116 and second shaft 1118 may cause movement of first arm 1112 and second arm 1114, respectively. Further, although shown as being semi-circular, it should be understood that first arm 1112 and/or second arm 1114 may have different shapes or sizes.

Medical systems, devices, and related methods discussed herein may be useful for performing a medical procedure, such as for manipulating tissue and increasing visibility or access to portions of a target site. Additionally, these medical systems, devices, and methods may help to allow for increasingly complex tissue manipulations or patterns, for example, by allowing independent control of one or more extendable arms. It should be understood that the embodiments discussed in this disclosure are exemplary in nature and should not be construed as limiting the scope of the invention. The described end cap designs, arm configurations, and actuation mechanisms may be modified or combined in various ways to suit specific medical applications or procedural requirements. Other variations and modifications of the described embodiments may be made without departing from the spirit and scope of this disclosure.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical system comprising:
an insertion device defining a working channel therein; and
an end cap device attachable to a distal portion of the insertion device, the end cap device comprising:
an end cap that includes a body defining a lumen aligned with the working channel; and
at least one arm movably coupled to the body, wherein the at least one arm is configured to transition between a retracted position and an extended position,
wherein, in the extended position, the at least one arm extends radially outward relative to a central longitudinal axis of the lumen.

2. The medical system of claim 1, wherein the at least one arm comprises a curved shape when in the extended position.

3. The medical system of claim 1, wherein the at least one arm comprises a plurality of arms arranged circumferentially around the lumen.

4. The medical system of claim 3, wherein each arm of the plurality of arms is independently movable between the retracted position and the extended position.

5. The medical system of claim 1, further comprising at least one spring coupled to the at least one arm, wherein the at least one spring is configured to bias the at least one arm toward one of the retracted position or the extended position.

6. The medical system of claim 1, further comprising at least one actuation wire coupled to the at least one arm, wherein the at least one actuation wire is configured to move the at least one arm between the retracted position and the extended position.

7. The medical system of claim 6, further comprising a pulley positioned distal to a proximal end of the at least one arm, wherein the at least one actuation wire interacts with the pulley to facilitate movement of the at least one arm.

8. The medical system of claim 1, wherein the body has an opening formed therethrough, and wherein the at least one arm is movable through the opening when moving between the retracted position and the extended position.

9. The medical system of claim 1, wherein the at least one arm has a variable cross-section along a longitudinal length of the at least one arm, wherein at least one distal portion of the at least one arm is wider than at least one proximal portion of the at least one arm.

10. The medical system of claim 1, wherein the at least one arm comprises a wire arm formed by a curved section of an actuation wire.

11. The medical system of claim 1, further comprising:
an actuation wire coupled to the at least one arm; and
an actuator apparatus coupled to a proximal end of the actuation wire, wherein the actuator apparatus is configured to control movement of the at least one arm.

12. The medical system of claim 11, wherein the actuator apparatus comprises:
a base having at least one slot; and
at least one control lever slidably disposed in the at least one slot,
wherein the proximal end of the actuation wire is movably coupled to the at least one control lever and a distal end of the actuation wire is movably coupled to the at least one arm.

13. The medical system of claim 12, wherein the at least one control lever comprises a plurality of control levers, and wherein the at least one slot comprises a plurality of slots configured to guide translation of the plurality of control levers received therein.

14. The medical system of claim 1, further comprising a medical device that is insertable through the insertion device and through the end cap device to a target site, wherein the medical device includes a handle having at least one actuator, a shaft extending distally from the handle, and a tool that is movable through the shaft to the target site.

15. The medical system of claim 14, wherein the tool is an electrosurgical knife.

16. A medical system comprising:
an insertion device defining a working channel; and
an end cap device attachable to a distal end of the insertion device, the end cap device comprising:
an end cap having a body defining a lumen substantially aligned with the working channel; and
a plurality of arms movably coupled to the body, wherein each arm of the plurality of arms is independently movable at least between a retracted position and an extended position.

17. The medical system of claim 16, further comprising an actuator apparatus operably connected to the plurality of arms and configured to independently control movement of each arm.

18. The medical system of claim 17, wherein the actuator apparatus comprises:
a plurality of control levers, each control lever being associated with one of the plurality of arms; and
a plurality of actuation wires, each actuation wire being coupled to one of the plurality of control levers, such that movement of the control levers imparts movement to the actuation wires to control a position of the arms.

19. A method for performing a medical procedure, comprising the steps:
inserting an insertion device into a body lumen to a target site, wherein an end cap device is attached to a distal end of the insertion device, wherein the end cap device includes an end cap having a body and at least one arm movably coupled to the body;
inserting a medical device through the insertion device to the target site;
cutting a target tissue at the target site via a tool of the medical device, wherein the tool is an electrosurgical knife; and
manipulating the target tissue with the end cap device, wherein manipulating the target tissue comprises distally extending the at least one arm from a retracted position toward an extended position distal of the end cap.

20. The method of claim 19, wherein distally extending the at least one arm comprises independently actuating at least one of a plurality of arms via an actuator apparatus, wherein the actuator apparatus includes a plurality of control levers movably coupled to the plurality of arms.
